(19) United States
(12) Patent Application Publication
(10) Pub. No.: US 2023/0099978 A1
(43) Pub. Date: Mar. 30, 2023

(54) CAPSULE FOR A DELIVERY APPARATUS FOR A PROSTHETIC MEDICAL DEVICE

(71) Applicant: Edwards Lifesciences Corporation, Irvine, CA (US)

(72) Inventor: David Maimon, Atlit (IL)

(21) Appl. No.: 18/075,939

(22) Filed: Dec. 6, 2022

Related U.S. Application Data
(63) Continuation of application No. PCT/US2021/036107, filed on Jun. 7, 2021.
(60) Provisional application No. 63/036,377, filed on Jun. 8, 2020.

(51) Int. Cl. A61F 2/24

(52) U.S. Cl. CPC A61F 2/2436; A61F 2240/001

(57) ABSTRACT

A capsule for a delivery apparatus and associated methods are disclosed. As one example, a capsule can include at least one end portion, arranged at a proximal end or distal end of the capsule, that narrows in diameter from a wider outer diameter of a middle portion of the capsule to a narrower outer diameter at an end of the at least one end portion, the end spaced away from the middle portion, where the at least one end portion is configured to expand to the wider outer diameter in response to a radially outward pressure from the prosthetic medical device during removal of the prosthetic medical device from inside of the capsule.

### CROSS REFERENCE TO RELATED APPLICATION

This application is a continuation of a PCT Application No. PCT/US2021/036107, filed Jun. 7, 2021, which claims the benefit of U.S. Provisional Patent Application No. 63/036,377, filed Jun. 8, 2020, where each of above-referenced applications is incorporated herein by reference in its entirety.

### FIELD

The present disclosure relates to a capsule for a delivery apparatus that is configured to reduce push forces experienced during advancing a prosthetic medical device, such as a prosthetic heart valve, contained within the capsule via the delivery apparatus to a target implantation site.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic valve reaches the implantation site in the heart. The prosthetic valve is then expanded to its functional size, for example, by deploying the prosthetic valve from a capsule of the delivery apparatus so that the prosthetic valve can self-expand to its functional size (or expand to its function size via a mechanical actuating mechanism).

In some embodiments, an introducer sheath may be inserted into a patient's vasculature to aid in delivery of the prosthetic heart valve (or other prosthetic medical device, such as a stent) to the target implantation site. For example, the delivery apparatus can be advanced through the sheath and the patient's vasculature (or other native anatomy) to the target implantation site. The capsule, arranged at a distal end of the delivery apparatus, typically is the widest portion of the delivery apparatus (the portion with the greatest diameter). The forces experienced by a user advancing the delivery apparatus through the introducer sheath and the patient's vasculature, due to friction created between an outer surface of the capsule and an inner surface of the sheath, for example, may be referred to as “push forces”. In some embodiments, the push forces may be larger for longer and larger diameter capsules. The capsule length and outer diameter may be dictated by a crimped length and diameter of the prosthetic heart valve (or other prosthetic medical device). It is desirable for the push forces to be reduced to enable easier advancement of the delivery apparatus to the target implantation site and reduce a likelihood of trauma to the patient's native anatomy.

Accordingly, a need exists for a delivery apparatus capsule that is configured to reduce the push forces experienced by a user during advancing the delivery apparatus through a patient's vasculature, to the target implantation site for the prosthetic medical device contained within the capsule.

### SUMMARY

Described herein are embodiments of a capsule of a delivery apparatus configured to deliver a prosthetic medical device to a target implantation site and reduce push forces experienced during advancing the delivery apparatus to the target implantation site, and related methods for using such a delivery apparatus and capsule to deliver the prosthetic medical device to the target implantation site. In some embodiments, the capsule can be configured to have a shorter effective length (e.g., the length of the portion of the capsule that can have contact with the inner wall of the sheath during advancement through a patient). In some embodiments, the shorter effective length is achieved by configuring the capsule to have one or more tapered ends that can be configured to expand during removal of the prosthetic medical device from the capsule at the target implantation site.

In one representative embodiment, a capsule of a delivery apparatus configured to deliver a prosthetic medical device to a target implantation site includes at least one end portion, arranged at a proximal end or distal end of the capsule, that narrows in diameter from a wider outer diameter of a middle portion of the capsule to a narrower outer diameter at an end of the at least one end portion, the end spaced away from the middle portion of the capsule, where the at least one end portion is configured to expand to the wider outer diameter in response to a radially outward pressure from the prosthetic medical device during removal of the prosthetic medical device from inside of the capsule.

In another representative embodiment, a delivery apparatus includes: an outer shaft; a nosecone coupled to a distal end of an inner shaft of the delivery apparatus, the inner shaft arranged within at least a portion of the outer shaft; and a capsule coupled to a distal end of the outer shaft, proximal to the nosecone, and configured to move axially relative to the nosecone, the capsule includes: a cylindrical middle portion that has a first outer diameter along its length; and an end portion configured to move between a first configuration where the end portion tapers radially inward from the middle portion so that the end portion has a reduced diameter relative to the outer diameter of the middle portion and a second configuration where the end portion is expanded and has an expanded diameter that is larger than the reduced diameter.

In one representative embodiment, a method includes: in response to and during actuating a capsule of a delivery apparatus to move axially away from a radially compressed prosthetic medical device arranged on a distal end portion of the delivery apparatus, in order to uncover the radially compressed prosthetic medical device, radially expanding a distal end portion of the capsule from a narrowed configuration where an outer diameter of a distal end of the distal end portion is smaller than an outer diameter of a middle portion of the capsule to an expanded configuration where the outer diameter of the distal end is equal to or greater than the outer diameter of the middle portion.

In another representative embodiment, a method includes: forming a tapered, narrower diameter portion of a capsule of a delivery apparatus at a distal end portion of the capsule, so that an outer diameter of the distal end portion narrows radially inward from a wider, middle portion of the capsule, the capsule arranged on a distal end portion of the delivery apparatus and configured to cover and retain a radially compressed prosthetic heart valve therein; and upon reaching a target implantation site for the prosthetic heart valve, retracting the capsule, in an axial direction, away from the prosthetic heart valve and radially expanding the distal end portion so that the outer diameter of the distal end portion is equal to or larger than a diameter of the middle portion of the capsule.

In another representative embodiment, a method includes: forming a tapered, narrower diameter portion of a capsule of a delivery apparatus at a distal end portion of the capsule, so that an outer diameter of the distal end portion narrows radially inward from a wider, middle portion of the capsule, the capsule arranged on a distal end portion of the delivery apparatus and configured to cover and retain a radially compressed prosthetic heart valve therein. The distal end portion of the capsule is configured to radially expand such that the outer diameter of the distal end portion, along a length of the distal end portion, is equal to or larger than a diameter of the middle portion of the capsule, in response to a radially outward pressure from the prosthetic heart valve during removal of the prosthetic heart valve from inside of the capsule.

The foregoing and other objects, features, and advantages of the invention will become more apparent from the following detailed description, which proceeds with reference to the accompanying figures.

### BRIEF DESCRIPTION OF THE DRAWINGS

FIG. 1 is a side elevation view of an exemplary embodiment of an implantable prosthetic heart valve that can be loaded into a capsule of a delivery apparatus using a loading apparatus, as disclosed herein.

FIG. 2 is a cross-sectional side view of an exemplary embodiment of a delivery apparatus for delivering a prosthetic heart valve, such as the prosthetic heart valve of FIG. 1, to a target implantation site, the delivery apparatus including a capsule for retaining the prosthetic heart valve therein in a compressed configuration.

FIG. 3 is a side elevation view of the distal end portion of the delivery apparatus of FIG. 2 shown with the capsule of the delivery apparatus advanced over a portion of the prosthetic heart valve frame.

FIG. 4 is a cross-sectional side view of an embodiment of a capsule of a delivery apparatus that has a longer effective length.

FIG. 5 is a cross-sectional side view of a first embodiment of a capsule of a delivery apparatus that has a reduced effective length due to a distal end that is configured to have a reduced diameter relative to a remainder of the capsule during advancing the delivery apparatus to a target implantation site in a patient.

FIG. 6 is a cross-sectional side view of a second embodiment of a capsule of a delivery apparatus that has a reduced effective length due to a distal end and proximal end that are both configured to have a reduced diameter relative to a remainder of the capsule during advancing the delivery apparatus to a target implantation site in a patient.

FIG. 7 is a side view of the first embodiment of the capsule of FIG. 5 showing a plurality of notches, forming wings, that allow the narrowed portion of the capsule to expand during removal of a prosthetic medical device from the capsule.

FIG. 8 is a side view of the second embodiment of the capsule of FIG. 6 showing a plurality of notches, forming wings, that allow the narrowed portion, at the distal end, of the capsule to expand during removal of a prosthetic medical device from the capsule.

FIG. 9 is a side view of the first embodiment of the capsule of FIG. 7, showing an expanded, distal end of the capsule during removal of the prosthetic medical device from the capsule.

FIG. 10 is a cross-sectional side view of the capsule of FIG. 5 with a nosecone arranged adjacent to and at least partially covering a distal end of the capsule.

FIG. 11 is a flow chart of a method for operating a capsule of a delivery apparatus configured to deliver a prosthetic medical device to a target implantation site in a patient, the capsule having a reduced effective length.

### DETAILED DESCRIPTION

### General Considerations

For purposes of this description, certain aspects, advantages, and novel features of the embodiments of this disclosure are described herein. The described methods, systems, and apparatus should not be construed as limiting in any way. Instead, the present disclosure is directed toward all novel and non-obvious features and aspects of the various disclosed embodiments, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present, or problems be solved.

Features, integers, characteristics, compounds, chemical moieties, or groups described in conjunction with a particular aspect, embodiment or example of the disclosure are to be understood to be applicable to any other aspect, embodiment or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The disclosure is not restricted to the details of any foregoing embodiments. The disclosure extends to any novel one, or any novel combination, of the features disclosed in this specification (including any accompanying claims, abstract, and drawings), or to any novel one, or any novel combination, of the steps of any method or process so disclosed.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatus can be used in conjunction with other systems, methods, and apparatus.

As used herein, the terms “a,” “an,” and “at least one” encompass one or more of the specified element. That is, if two of a particular element are present, one of these elements is also present and thus “an” element is present. The terms “a plurality of” and “plural” mean two or more of the specified element.

As used herein, the term “and/or” used between the last two of a list of elements means any one or more of the listed elements. For example, the phrase “A, B, and/or C” means “A,” “B,” “C,” “A and B,” “A and C,” “B and C,” or “A, B, and C.”

As used herein, the term “coupled” generally means physically coupled or linked and does not exclude the presence of intermediate elements between the coupled items absent specific contrary language.

Directions and other relative references (e.g., inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part can become a “lower” part simply by turning the object over. Nevertheless, it is still the same part and the object remains the same. As used herein, “and/or” means “and” or “or,” as well as “and” and “or.”

As used herein, with reference to the prosthetic medical device (e.g., heart valve), capsule, and the delivery apparatus, “proximal” refers to a position, direction, or portion of a component that is closer to the user and/or a handle of the delivery apparatus that is outside the patient, while “distal” refers to a position, direction, or portion of a component that is further away from the user and/or the handle of the delivery apparatus and closer to the implantation site. The terms “longitudinal” and “axial” refer to an axis extending in the proximal and distal directions, unless otherwise expressly defined. Further, the term “radial” refers to a direction that is arranged perpendicular to the axis and points along a radius from a center of an object (where the axis is positioned at the center, such as the longitudinal axis of the prosthetic valve).

### Examples of the Disclosed Technology

Described herein are examples of a capsule for a delivery apparatus that has a delivery configuration where one or more ends of the capsule are narrowed relative to a remainder of the capsule in order to reduce an outer surface area of the capsule that can have contact with an inner surface of a sheath through which the delivery apparatus is advanced, to reach a target implantation site for a prosthetic medical device radially compressed inside the capsule. As such, friction between the capsule and sheath (and/or native anatomy of the patient) may be reduced, thereby reducing push forces experienced by a user advancing the delivery apparatus through the patient. In some embodiments, a narrowed, distal end of the capsule can be configured to expand to a wider diameter to enable removal of the prosthetic medical device from the capsule, upon reaching the target implantation site.

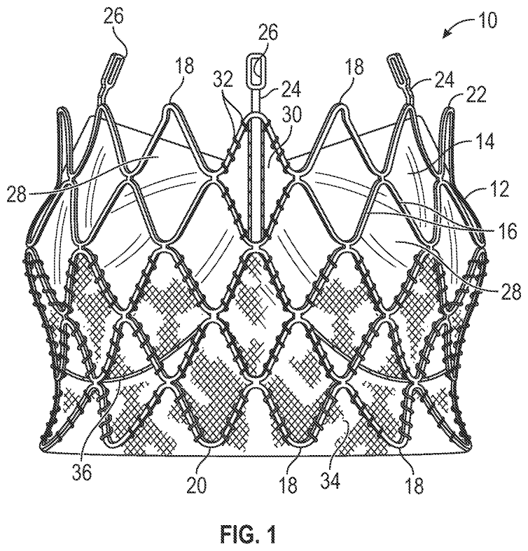

In some embodiments, the delivery apparatus is configured to deliver and implant a prosthetic heart valve, such as the example prosthetic heart valve of FIG. 1, at a selected implantation site within a patient (e.g., within the native aortic valve, mitral valve, tricuspid valve or pulmonary valve). For example, by retracting the capsule of the delivery apparatus away from the radially compressed prosthetic heart valve, the prosthetic heart valve can expand to is expanded configuration and implant itself at the target implantation site. In alternate embodiments, upon retracting the capsule away from the radially compressed prosthetic heart valve, an additional actuator of the delivery apparatus can be actuated to mechanically expand the prosthetic heart valve to its expanded configuration, to implant the valve at the target implantation site.

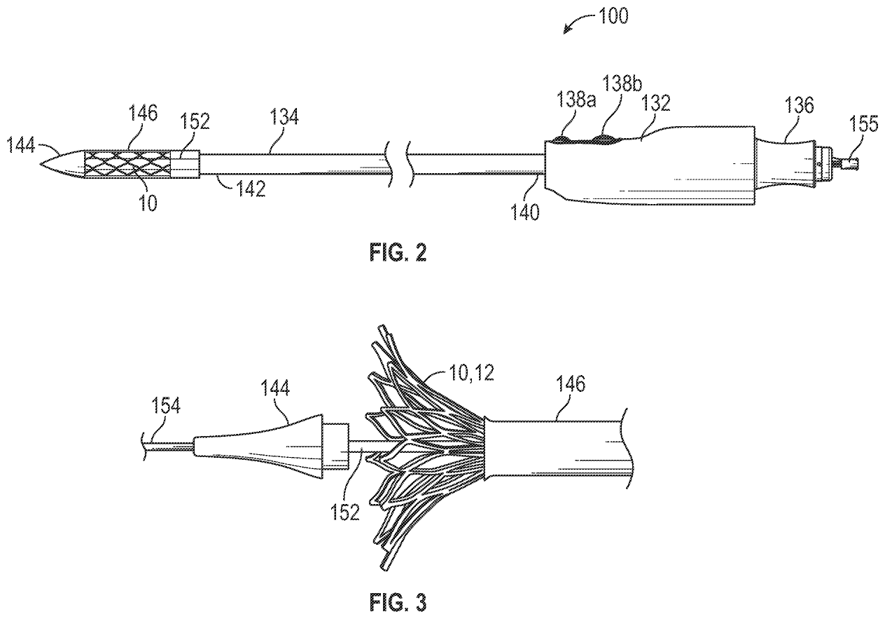

FIG. 1 shows a prosthetic heart valve 10, according to one embodiment, that can be implanted with a delivery apparatus, such as the delivery apparatus 100 of FIG. 2 (as described further below). In some embodiments, the prosthetic heart valve 10 is a self-expanding valve that is delivered in a radially compressed state to a deployment site via the delivery apparatus 100. When advanced from a delivery capsule at the distal end of the delivery apparatus (FIG. 2), the prosthetic valve can radially self-expand to its functional size.

The prosthetic heart valve 10 comprises a stent, or frame 12 and a valvular structure 14 (e.g., leaflets or a flap valve) supported by the frame 12. The frame 12 can have a plurality of interconnected struts 16 arranged in a lattice-like pattern and forming a plurality of apices 18 at the inflow and outflow ends 20, 22, respectively, of the frame 12

The frame 12 can include a plurality of angularly-spaced posts 24 extending from respective apices 18 at the outflow end of the frame 12. The frame 12 in the illustrated embodiment includes three such posts 24, although a greater or fewer number of posts can be used. In one implementation, the frame 12 can have posts extending from all the apices 18 at the outflow end of the frame. Each post 24 can have an eyelet or aperture 26, which can be used to form a releasable connection with the delivery apparatus 100.

In some embodiments, the frame 12 can be without posts 24 and apertures 26 can be formed in the apices 18 at the outflow end of the frame.

In other embodiments, the apertures 26 (whether formed in posts 24 or in the apices 18) can be formed at the inlet (or inflow) end 20 of the frame 12 where other delivery apparatus configurations or other delivery techniques require apertures at the inlet end of the frame, such as a transapical delivery approach.

In particular embodiments, the prosthetic heart valve 10 is a self-expandable heart valve wherein the frame 12 is a made of a super-elastic, self-expanding material (e.g., a nickel titanium alloy such as Nitinol), as is known in the art. When used with the delivery apparatus 100 (FIG. 2), the prosthetic valve 10 can self-expand from a radially compressed state to a radially expanded state when advanced from a delivery capsule (e.g., a delivery sheath) of the delivery apparatus.

In other embodiments, the frame 12 can be made of any of various suitable plastically-expandable materials (e.g., stainless steel, cobalt-chromium alloy, etc.) and the prosthetic heart valve can be expanded from a radially compressed state to a radially expanded state by actuating other expansion means of the delivery apparatus that produces radial expansion of the prosthetic valve, such as an inflatable balloon.

The valvular structure 14 can comprise a plurality of leaflets 28. The valvular structure typically comprises three leaflets 28 arranged in a tricuspid arrangement, although a greater or fewer number of leaflets 28 can be used. The leaflets 28 can be made of any various suitable materials, including natural tissue (e.g., bovine pericardium or pericardium from other sources) or synthetic materials (e.g., polyurethane). Adjacent side portions at the outflow edges (the upper edges in the drawings) of adjacent leaflets can be secured to each other to form commissures 30 of the valvular structure, which can be secured to the frame with sutures 32.

The prosthetic valve 10 can further include an inner skirt 34 mounted on the inside of the frame 12. The skirt 34 helps establish a seal with the surrounding tissue after implantation. The skirt 34 can also be used to mount portions of the leaflets 28 to the frame 12. For example, in the illustrated embodiment, the inflow edges of the leaflets (the lower edges in the drawings) can be sutured to the skirt 34 along suture line 36. The skirt 34 can be connected directly to the frame 12, such as with sutures. Although not shown, the prosthetic valve 10 can include an outer skirt mounted on the outside of the frame in lieu of or in addition to the inner skirt 34 to further seal the prosthetic valve against surrounding tissue. The inner and/or outer skirts can be made of any of various suitable materials, including natural tissue (e.g., pericardium tissue) or any of various synthetic materials, which may be woven, non-woven, braided, knitted, and/or combinations thereof. In one specific implementation, the inner skirt 34 is made of a polyethylene terephthalate (PET) fabric.

Exemplary configurations of the prosthetic heart valve are further disclosed in U.S. Patent Application Publication Nos. 2014/0343670, 2012/0123529, 2010/0036484, and 2010/0049313, the disclosures of which are incorporated herein by reference.

Prosthetic heart valve 10, or another type of prosthetic heart valve (e.g., a mechanically expandable valve) or implantable, expandable medical device, such as an expandable stent, can be delivered to an implantation site via a delivery apparatus, an embodiment of which is shown at FIGS. 2 and 3 as delivery apparatus 100.

In an exemplary embodiment shown in FIG. 2, the delivery apparatus 100 can comprise a handle portion 132 and a first shaft 134 extending distally therefrom. A user, such as a physician or clinician, can operate the delivery apparatus 100 via actuation of a plurality of knobs 136, dials, and/or buttons 138a, 138b located on the handle portion 132. The first shaft 134 has a proximal end portion 140 and a distal end portion 142. The proximal end portion 140 of the first shaft 134 can be coupled to the handle portion 132.

As shown in FIG. 2, the delivery apparatus 100 can include a second shaft 152. The second shaft 152 extends distally from the handle portion 132 and co-axially though the first shaft 134. In the illustrated embodiment, the first shaft 134 is the outermost shaft of the delivery apparatus and therefore can be referred to as the outer shaft 134 of the delivery apparatus. In the illustrated embodiment, the second shaft 152 is the innermost shaft of the delivery apparatus and therefore can be referred to as the inner shaft 152 of the delivery apparatus. In some embodiments, the delivery apparatus 100 may include a third shaft that is an intermediate shaft, arranged between the inner shaft 152 and the outer shaft 134.

A nosecone 144 can be connected to or mounted on a distal end portion of the inner shaft 152. The nosecone 144 can have a tapered outer surface as shown for atraumatic tracking of the delivery apparatus 100 through a patient's vasculature. The inner shaft 152 extends through the prosthetic valve 10.

In certain embodiments, the first and second shafts 134 and 152, respectively, can be configured to be moveable relative to each other, including relative axial movement (in the proximal and distal directions) and/or relative rotational movement (in the clockwise and counterclockwise directions). A guide wire 154 (as shown in FIG. 3) can extend through the central lumen of the inner shaft 152 and the inner lumen of the nosecone 144 so that the delivery apparatus 100 can be advanced over the guide wire 154 inside the patient's vasculature during delivery of the prosthetic valve 10. The guide wire 154 may be inserted into the inner shaft 152 via a proximal port 155 of the handle portion 132 (FIG. 2).

A delivery capsule 146 is coupled to the distal end portion 142 of the first shaft 134, proximal to the nosecone 144. The delivery capsule 146 houses the prosthetic valve 10 therein, in a radially compressed state, as shown at FIGS. 2-3. In one embodiment, the delivery capsule 146 covers and retains the underlying compressed prosthetic valve of FIG. 1. In some embodiments, the delivery apparatus 100 is particularly suited for delivering and implanting a self-expandable prosthetic valve 10 that radially expands to its functional size under its own resiliency when deployed from the delivery capsule 146. The prosthetic valve 10 can be deployed from the capsule 146 by retracting the capsule 146 proximally relative to the prosthetic valve and/or advancing the prosthetic valve 10 distally relative to the capsule 146 so that the prosthetic valve is advanced out of the open distal end of the capsule. In some embodiments, the capsule 146 can be retracted by rotating an inner torque shaft of the delivery apparatus, which produces axial movement of the capsule 146 relative to the outer shaft 134. In other embodiments, the capsule 146 can be an extension of the outer shaft 134 or can be affixed to the outer shaft 134 such that axial movement of the outer shaft (distally or proximally) produces axial movement of the capsule in the same direction. Further details of delivery apparatuses that can be used for deploying self-expanding prosthetic valves can be found in U.S. Patent Application Publication Nos. 2014/0343670, 2012/0239142 and 2010/0049313, which are incorporated herein by reference.

In other embodiments, the delivery apparatus 100 can deliver another type of prosthetic heart valve, such as a mechanically expandable heart valve. Various embodiments of mechanically expandable prosthetic heart valves, actuator assemblies, and delivery apparatuses for delivering such mechanically expandable valves can be found in U.S. Patent Application Publication Nos. 2018/0153689, 2018/0344456, and 2019/0060057 and International Patent Application Nos. PCT/US2020/057691, PCT/US2020/063104, and PCT/US2021/022467, each of which is incorporated by reference herein in their entireties.

As shown at FIG. 2, the delivery capsule 146 is configured to accommodate the prosthetic heart valve 10, or another type of prosthetic heart valve or implantable medical device, in a radially compressed state for delivery into a patient's vasculature. FIG. 3 shows the delivery capsule 146 advanced over a portion of the prosthetic heart valve 10.

In some embodiments, a valve-retaining mechanism can be used to form a releasable connection between the prosthetic valve 10 and the delivery apparatus 100. For example, in some embodiments, the posts 24 of the frame 12 can be retained in corresponding recesses of a shaft or retaining member of the delivery apparatus, which allow the posts of the frame to expand out of their corresponding recesses when the capsule 146 is retracted to deploy the prosthetic valve. In other embodiments, a plurality of suture loops can be connected to a suture retention mechanism of the delivery apparatus and can extend through the apertures 26 of posts 24 to form a releasable connection between the prosthetic valve and the delivery apparatus. In other embodiments, the retaining mechanism can comprise inner and outer metal fork members that form a release connection between the delivery apparatus and the prosthetic valve. Further details regarding the delivery apparatus, including alternative valve-retaining mechanisms, are disclosed in U.S. Patent Application Publication Nos. 2014/0343670, 2012/0239142 and 2010/0049313.

FIG. 4 is a schematic of a first embodiment of a capsule 202 arranged on a distal end portion of an outer shaft 204 of a delivery apparatus 200. In some embodiments, the delivery apparatus 200 can be the same as or similar to the delivery apparatus 100 shown in FIG. 2. As such, the outer shaft 204 can be similar to the first shaft 134 of delivery apparatus 100 and the capsule 202 can be similar to capsule 146 shown in FIG. 2. As described above with reference to FIGS. 2 and 3, the capsule 202 encloses a radially compressed (e.g., crimped) prosthetic medical device 206 therein, such as the valve 10 of FIG. 1, another type of prosthetic heart valve (e.g., a mechanically expandable valve), or another type of radially expandable medical device (e.g., a stent).

A geometry of the capsule 202, including the outer diameter 208 and length 210, may be determined by a crimp profile of the radially compressed prosthetic medical device 206. For example, as shown in FIG. 4, the length 210 may correspond to (e.g., be the same or longer than) a length of the crimped prosthetic medical device 206. Further, an inner diameter 212 of the capsule 202 is dictated by the crimped diameter of the radially compressed prosthetic medical device 206. Thus, the outer diameter 208 of the capsule 202 can be selected based on the determined inner diameter 212 and a thickness of the capsule 202. Capsule 202 may represent a conventional capsule that has a relatively uniform outer diameter 208 along its entire length 210.

During an implantation procedure, the radially compressed prosthetic medical device 206, arranged on the distal end portion of the delivery apparatus 200, is advanced through the patient's vasculature (or other inner lumen) to a target implantation site, via the delivery apparatus. In some embodiments, an introducer sheath may be inserted into the patient's vasculature and the delivery apparatus 200 can be advanced through the sheath and the patient's vasculature to the target implantation site. During delivery of the device 206 to the target implantation site, the capsule 202 may contact (and slide against) the surrounding, inner walls of the sheath, resulting in friction between the capsule and sheath. These frictional forces between an outer wall of the capsule 202 and an inner wall of the sheath may be experienced by a user advancing the delivery apparatus as “push forces”. For example, as the friction between the capsule 202 and sheath increases, the push forces felt by a user pushing the delivery apparatus through the sheath, may also increase. In some embodiments, the frictional and push forces may increase as a surface contact area between the outer wall (or surface) of the capsule 202 and inner wall (or surface) of the sheath increases. The surface contact area may be dictated by an effective length of the capsule 202. As used herein, “effective length” refers to the length of the portion of the capsule 202 that can contact the inner wall of the sheath during an implantation procedure. As shown in FIG. 4, the effective length of the capsule 202 is equal to the total length 210 of the capsule 202.

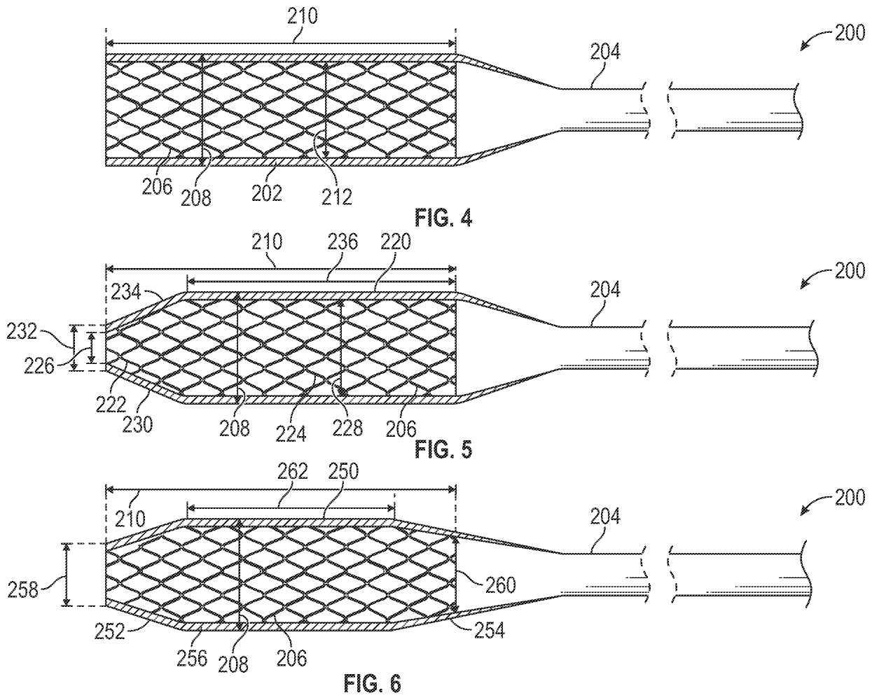

In some embodiments, the crimp profile of the radially compressed prosthetic medical device 206 may not have a constant diameter along its length. For example, the device 206 may have portions at its distal and/or proximal ends that have a smaller diameter than in a middle portion of the device and/or taper radially inward from the middle portion, when in the radially compressed state (e.g., as shown in FIGS. 5 and 6, as described further below).

Thus, in these embodiments, it may be possible for the capsule to assume a narrowed shape with a smaller diameter around these smaller diameter portions of the device 206, during delivery of the device 206 to the target implantation site. As a result, the effective length of the capsule may be reduced, thereby reducing the surface area of the capsule that can come into contact with the sheath and reducing the push forces experienced by the user. As a result, the delivery apparatus may be more easily navigated through the patient's vasculature.

FIGS. 5-10 show different embodiments of a capsule that has a reduced effective length as a result of proximal and/or distal ends that can be crimped down or arranged into a smaller diameter (relative to a remainder or middle portion of the capsule) during delivery of a prosthetic medical device arranged within the capsule to its target implantation site. Specifically, FIGS. 5, 7, 9, and 10 show a first embodiment of a capsule 220 including a distal end (or distal end portion) that is configured to be crimped or arranged into a smaller diameter configuration than a remainder of the capsule 220 for delivery of the device 206 to the target implantation site (e.g., during advancing the delivery apparatus to the target implantation site). FIGS. 6 and 8 show a second embodiment of a capsule 250, which may be similar to the capsule 220, but both its distal end and proximal end are configured to be crimped or arranged into a smaller diameter configuration than a remainder (e.g., middle portion) of the capsule 250 for delivery of the device 206 to the target implantation site.

Turning first to FIG. 5, the capsule 220 is shown in a delivery configuration. The delivery configuration may be defined herein as the configuration of the capsule 220 during advancing the distal end portion of the delivery apparatus 200 through an introducer sheath and the patient's vasculature (or other inner lumen), to deliver the device 206 to the target implantation site (e.g., the patient's heart). In the delivery configuration, the device 206 is arranged on the distal end portion of the delivery apparatus 200, in its radially compressed configuration, and the capsule 220 is arranged over (covering) the radially compressed device 206.

As shown in FIG. 5, in some embodiments, when in its radially compressed configuration, the device 206 can have a distal end portion 222 that has a smaller diameter than a remainder of the device 206. For example, as shown in FIG. 5, the distal end portion 222 can taper (or in alternate embodiments, step) radially inward from a wider, middle portion (or remainder) 224 of the device 206. As a result, a distal end of the device 206 has a first diameter 226 that is smaller than a second diameter 228 of the middle portion 224. In some cases, the distal end portion 222 can be crimped to a greater extent than the middle portion 224 by virtue of there being less material (e.g., skirt or leaflet material) located within the distal end portion, thereby allowing the device 206 to assume a tapered shape along the distal end portion 222.

Since at least a portion of the device 206 (e.g. the distal end portion 222) has a smaller diameter than the middle portion 224 of the device 206, it is not necessary for the capsule 220 to be completely cylindrical and have a same diameter (e.g., outer diameter 208) along an entire length 210 of the capsule 220. Instead, as shown in FIG. 5, a distal end portion 230 of the capsule 220, which corresponds to and is arranged around the distal end portion 222 of the device 206, can be narrower than a remainder of the capsule 220 when in the delivery configuration.

For example, as shown in FIG. 5, the distal end portion 230 of the capsule 220 tapers from the outer diameter 208 to a narrower diameter 232 at its distal end. In alternate embodiments, the taper of the distal end portion 230 of the capsule 220 may be more or less pronounced than shown in FIG. 5, or the distal end portion may step down to a more constant smaller diameter, based on (e.g., matching, in some embodiments) the profile of the distal end portion 222 of the device 206. As shown in FIG. 5, the distal end portion 230 has an outer diameter that is smaller than the outer diameter 208 of the remainder of the capsule 220. As a result, the outer surface 234 of the capsule 220, in the region of the distal end portion 230, may not come into contact with (or have at least reduced contact with) inner walls of the introducer sheath and/or the patient's anatomy (e.g., vasculature). Thus, the effective length 236 of the capsule 220 is smaller than the total length 210 of the capsule 220 (and the effective length of the conventional capsule 202 which has a relatively constant diameter along its length 210 and can be cylindrical in shape). By having a narrower diameter portion (e.g., at its distal end), the capsule 220 has a smaller effective length 236, and thus, push forces experienced during advancing the delivery apparatus 200 through the patient's vasculature (and through the sheath) may be reduced (as compared to the conventional capsule 202 of FIG. 4).

In alternate embodiments, a different or additional portion of the capsule may have a reduced diameter relative to a remainder or middle portion of the capsule, thereby reducing the effective length of the capsule. For example, as shown in FIG. 6, when in the delivery configuration, the capsule 250 has both a distal end portion 252 and a proximal end portion 254 that have smaller outer diameters relative to a remainder or middle portion 256 of the capsule 250. This may be possible due to the device 206 having both a distal end portion and a proximal end portion that are tapered or have a reduced diameter compared to a middle portion of the device 206.

For example, as shown in FIG. 6, a first outer diameter 258 of a distal end of the capsule 250 (end of distal end portion 252) and a second outer diameter 260 of a proximal end (end of proximal end portion 254) are each smaller than the outer diameter 208 of the middle portion 256 of the capsule 250. As a result, the capsule 250 has an effective length 262 which is smaller than the total length 210 of the capsule 250. In some embodiments, the effective length 262 of the capsule 250 is smaller than the effective length 236 of the capsule 220. As a result, the delivery apparatus 200 with the capsule 250 may result in reduced push forces during delivery of the device 206 to the target implantation site than a delivery apparatus including capsule 220 (FIG. 5) or capsule 202 (FIG. 4).

In some embodiments, the amount of tapering, the smallest outer diameter (e.g., outer diameters 258 and 260), or the length of the reduced diameter portions of the capsule 250 may be the same or different for the distal end portion 252 and the proximal end portion 254.

In alternate embodiments, a capsule, similar to capsule 220 and/or capsule 250 may only have a reduced diameter portion at its proximal end (e.g., only proximal end portion 254 has the reduced diameter relative to the outer diameter 208). As explained above, the size (e.g., diameter, length, and/or taper angle) of the reduced diameter portions of the capsule may be determined by a shape or profile of the device 206 in it radially compressed configuration. Thus, if a device 206 narrows at both its distal and proximal ends in the radially compressed state, then the capsule of the delivery apparatus may resemble capsule 250 of FIG. 6 having both narrowed proximal and distal end portions. In another example, if the device 206 narrows at only one of its ends (e.g. its distal end), then the capsule of the delivery apparatus may resemble capsule 220 of FIG. 5, having a single narrowed end (e.g., distal end portion 230). Further, a degree of angling or amount of narrowing of the distal and/or proximal end portions of the capsule may be selected based on a shape or profile of the corresponding distal and/or proximal end portions of the radially compressed device 206. In this way, the taper of the reduced diameter portion(s) of the capsule can be adapted to follow a general profile of the corresponding end portions of the device 206.

In some embodiments, a capsule having a smaller effective length than total length (e.g., capsule 220 of FIG. 5 and capsule 250 of FIG. 6) can be configured to have the smaller effective length in the delivery configuration, when the device 206 is arranged therein, but have a widened distal end portion (e.g., the distal end portion having an outer diameter that is the same or larger than the outer diameter 208) when loading the device 206 into the capsule and when removing the device from the capsule (e.g., upon reaching the target implantation site). For example, prior to loading the device 206 into the capsule and after uncovering the device 206 at the target implantation site by moving the device 206 away from the capsule or retracting the capsule away from the device 206, the capsule may assume a configuration similar to the capsule 202 of FIG. 4.

In some embodiments, after loading the device 206 into the capsule, the distal end portion and/or proximal end portion of the capsule may be crimped, formed, or set into the reduced diameter, delivery configuration (e.g., as shown in FIG. 5 or FIG. 6). In this way, the capsule (e.g., capsule 220 or capsule 250) can be configured to have one or more reduced diameter (or tapered) ends in the delivery configuration and expand, at least at the distal end, to a wider diameter during insertion and removal of the device into and from, respectively, the capsule.

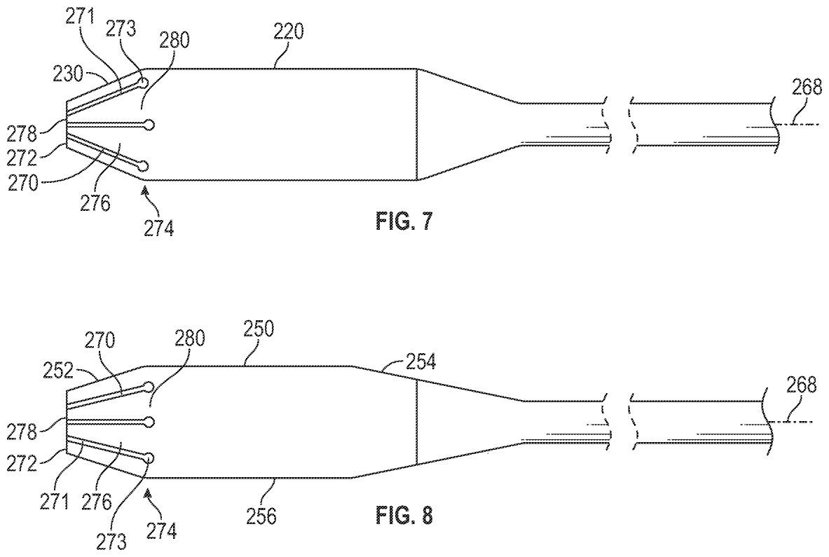

In some embodiments, as shown in the side views of FIGS. 7 and 8, the distal end portion 230 of the capsule 220 and the distal end portion 252 of the capsule 250 can include a plurality of elongate notches (or slots) 270 spaced apart from one another around a circumference of the distal end portion. Each notch 270 can extend from a distal end 272 of the capsule to a portion of the capsule that transitions from the distal end portion to the remainder or middle portion of the capsule (e.g., that has the larger outer diameter 208). The location of the proximal end the notches 270 at this portion of the capsule is labeled as 274 in FIGS. 7 and 8.

In some embodiments, each notch 270 extends through an entire thickness of the capsule.

The notches 270 form a plurality of wings (or wedges) 276 of the capsule. For example, each wing 276 can be formed between two adjacent notches 270. Each wing 276 can extend from the distal end 272 to the location 274. In this way, the wings 276 can be axially orientated, relative to a central longitudinal axis 268 of the capsule and delivery apparatus 200.

In some embodiments, the notches 270 can be regularly spaced apart from one another around the circumference of the capsule. In alternate embodiments, the notches 270 may be irregularly spaced around the circumference of the capsule. As a result, the wings 276 can have a same or different size and shape, based on the spacing of the notches 270.

In some embodiments, as shown in FIGS. 7 and 8, each notch 270 can include an elongate end 271, extending from the distal end 272 to the location 274 and a more bulbous end 273 arranged at the location 274 (e.g., at its proximal end). In some embodiments, by having a proximal end that is wider than the elongate end of the notch 270, the wings formed by the notches can have increased flexibility (e.g., increased ability to expand and/or compress radially).

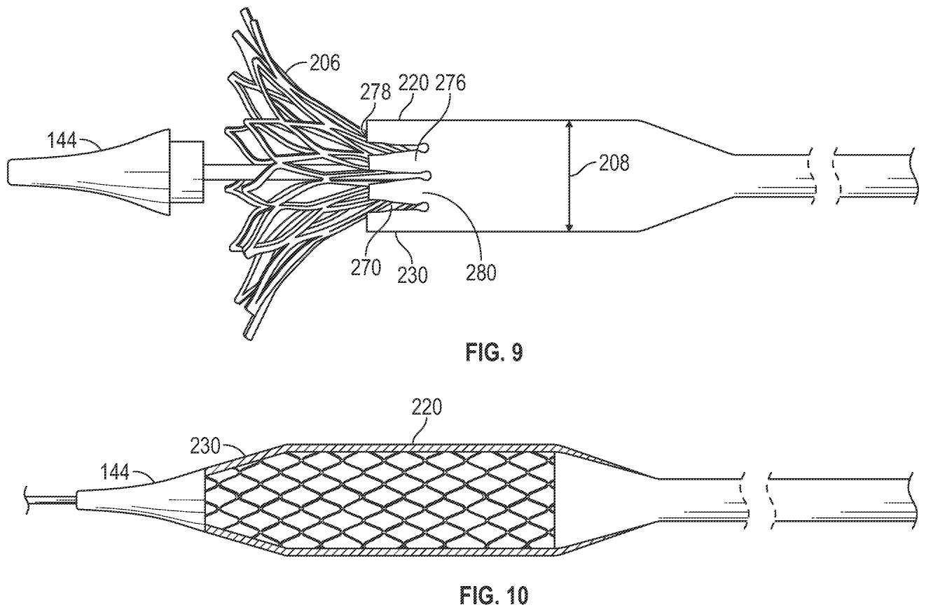

Prior to crimping or arranging the capsule (e.g., capsule 220 or capsule 250) into the delivery configuration (as shown in FIGS. 5-8), where the distal end portion and/or proximal end portion are narrowed relative to a remainder or middle portion of the capsule, the distal end portion of the capsule may resemble the capsule in FIG. 4 or 9. In this pre-delivery (and crimping) configuration, the wings 276 can be expanded radially outward at the distal end 272 such that the diameter of the capsule at the distal end 272 is the same, similar to, or larger than the outer diameter 208 of the remainder of the capsule.

In some embodiments, when the capsule includes a narrowed proximal end, as shown in the capsule of FIGS. 6 and 8, the proximal end portion 254 may remain in the narrowed configuration prior to crimping the capsule, after crimping the capsule, and after deploying the prosthetic medical device from the capsule. In this way, in some embodiments, the narrowed proximal end portion 254 may not change shape/diameter since the narrowed shape does not affect device loading into or removal from the capsule 250. Thus, in some embodiments, only the distal end portion can include wings 276 and notches 270 and the proximate end portion, even if configured to have a narrowed configuration, does not include wings and notches (e.g., as shown in the embodiment of FIG. 8)

In alternate embodiments, the narrowed proximal end can be configured to expand during removal of the device from the capsule.

After loading the prosthetic medical device (e.g., device 206 which in some embodiments can be a prosthetic heart valve) into the capsule (e.g., capsule 220 or 250), the distal end portion 230 of the capsule 220 or the distal end portion 252 of the capsule 250 can be crimped, compressed, or formed down into its tapered or reduced diameter, delivery configuration (as shown in FIGS. 5-8). In some embodiments crimping or compressing the distal end portion down into the reduced diameter, delivery configuration can include pressing the wings 276 radially inward, toward the central longitudinal axis 268 and forming the tapered (or inclined or smaller diameter) profile at the distal end portion 230 or 252. As such, in the delivery configuration, each wing 276 can angle radially inward from a wider, proximal end 280 of the wing 276 to a narrower, distal end 278 of the wing 276. The distal ends 278 of the plurality of wings 276 can form the distal end 272 of the capsule.

In some embodiments, the capsule 220 or 250 can comprise an elastic, self-expanding (e.g., shape memory) metallic or polymeric material (e.g., a nickel titanium alloy such as Nitinol) as is known in the art. For example, the notches 270 can be created in the distal end portion 230 or 252 of a cylindrical capsule (e.g., the expanded profile, as shown in FIG. 4), thereby forming the wings 276. The wings 276 can then be crimped radially inward and shape set (e.g., via heat setting or another forming method) in the reduced diameter, delivery configuration (as shown in FIGS. 5-8). Alternatively, the capsule 220 or 250 can comprise a plastically deformable material (e.g., stainless steel or a cobalt chromium alloy) and the wings 276 can be plastically deformed by crimping to assume the reduced diameter, delivery configuration.

In other embodiments, the capsule 220 or 250 can comprise an elastomeric material (e.g., silicone) which is formed into the conical, tapered, or reduced diameter shape shown in any of FIGS. 5-8. In this embodiment, the capsule 220 or 250 may not include the notches 270. Alternatively, in some embodiments, the elastomeric capsule 220 or 250 may include grooves or score lines, in the place of the notches 270, that form thinner portions of the distal end portion of the capsule (e.g., thinner, axially-extending grooves) that increase the flexibility of the distal end portion of the capsule. In other embodiments, the elastomeric capsule 220 or 250 may not include grooves, score lines, or notches, and instead the reduced diameter portion of the capsule (e.g., the distal end portion) may be configured to expand radially outward to the expanded configuration. For example, in some embodiments, the distal end portion of the capsule can comprise a thinner elastomeric material than a remainder of the capsule. Then, upon deploying the prosthetic medical device from the capsule, as explained further below, the elastomeric, distal end portion 230 or 252 of the capsule can expand (or stretch) radially outward, in response to the force of the prosthetic heart valve sliding axially outward from and through the distal end 272 of the capsule (e.g., as shown in FIG. 9).

Turning to FIG. 9, when the capsule 220 (or in alternate embodiments, capsule 250) is retracted away from the device 206 (or the device 206 is moved distally outward from the capsule) during the implantation procedure (e.g., after reaching the target implantation site), the wings 276 expand radially outward from the radially outward force of the device 206 against the inner walls (or inner surfaces) of the wings 276. As a result, the wings 276 of the distal end portion 230 open back up to the larger outer diameter 208 of the remainder of the capsule 220. As shown in FIG. 9, the notches 270 widen at their distal ends to accommodate this expansion.

In some embodiments, as shown in FIG. 10, when in the delivery configuration, the capsule 220 (or in alternate embodiments, capsule 250) can be arranged adjacent to and partially inside an interior of the nosecone 144 of the delivery apparatus. For example, the distal end of the narrowed distal end portion 230 can be inserted into an opening within the nosecone, forming a relatively continuous outer surface between the nosecone 144 and the capsule 220. This configuration, during delivery of the prosthetic medical device to the target implantation site, may result in a more atraumatic configuration. As a result, degradation to the introducer sheath and/or patient's anatomy during advancing the distal end portion of the delivery apparatus to the target implantation site may be reduced.

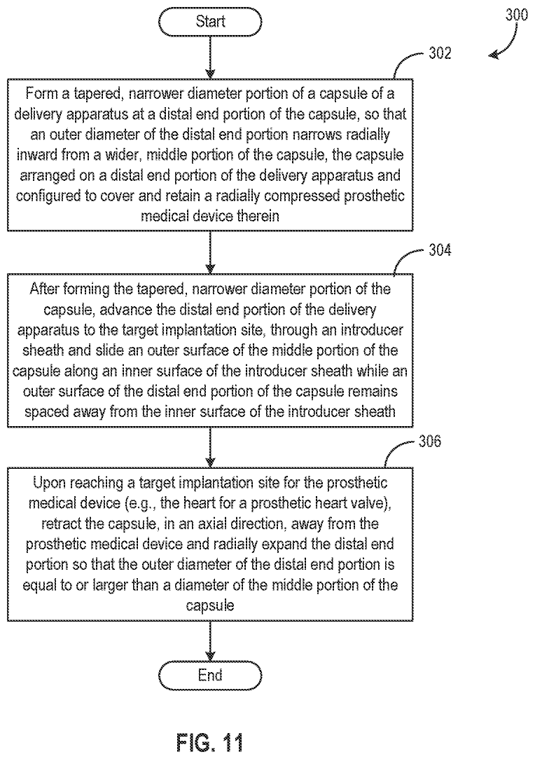

FIG. 11 illustrates a flow chart of a method 300 for operating a capsule of a delivery apparatus configured to deliver a prosthetic medical device (e.g., prosthetic heart valve) to a target implantation site in a patient. In some embodiments, the capsule can have a reduced effective length during delivery of the prosthetic medical device to the target implantation site (e.g., as shown in FIGS. 5-10).

The method 300 begins at 302 and includes forming a tapered, narrower diameter portion of a capsule of a delivery apparatus at a distal end portion of the capsule, so that an outer diameter of the distal end portion narrows radially inward from a wider, middle portion of the capsule, the capsule arranged on a distal end portion of the delivery apparatus and configured to cover and retain a radially compressed prosthetic medical device therein. In some embodiments, the method at 302 may including forming the tapered, narrower diameter distal end portion of the capsule after loading the radially compressed prosthetic medical device into the capsule. Thus, during loading of the device into the capsule, the distal end portion may be an a radially expanded configuration.

In some embodiments, at 302, forming the tapered, narrower diameter portion of the capsule includes moving distal ends of a plurality of wings forming the distal end portion of the capsule radially inward so that they are arranged proximate to one another and form the narrower diameter portion of the capsule, wherein each wing of the plurality of wings is separated from adjacent wings of the plurality of wings by an axially-extending notch in the distal end portion. The method at 302 can further include narrowing a plurality of notches arranged between and separating the plurality of wings, where each notch of the plurality of notches is arranged between two adjacent wings of the plurality of wings, and where radially expanding the distal end portion includes widening each notch of the plurality of notches.

In some embodiments, forming the tapered, narrower diameter portion of the capsule at 302 includes compressing the distal ends of the plurality of wings radially inward into a radially compressed configuration and setting the wings in the radially compressed configuration such that they are maintained in the radially compressed configuration until a radially outward force is applied to inner surfaces of the plurality of wings by the prosthetic heart valve, during the retracting the capsule.

In alternate embodiments, forming the tapered, narrower diameter portion of the capsule at 302 includes allowing the distal ends of the plurality of wings to retract radially inward into a non-deformed, resting state and, in response to retracting the capsule away from the prosthetic heart valve, elastically deforming the plurality of wings into an expanded state where distal ends of the plurality of wings are spaced further apart from one another around a circumference of the distal end portion.

At 304, the method includes, after forming the tapered, narrower diameter portion of the capsule, advancing the distal end portion of the delivery apparatus to the target implantation site, through an introducer sheath and sliding an outer surface of the middle portion of the capsule along an inner surface of the introducer sheath while an outer surface of the distal end portion of the capsule remains spaced away from the inner surface of the introducer sheath.

In one example, for implanting the prosthetic valve within the native aortic valve via a transfemoral delivery approach, the introducer sheath is first inserted into a femoral artery. The distal end portion of the delivery apparatus and the prosthetic valve are advanced through the introducer sheath, into and through the descending aorta, around the aortic arch toward the native aortic valve. In some examples, the delivery apparatus is manipulated to position the prosthetic valve within the native aortic valve.

At 306, the method include, upon reaching a target implantation site for the prosthetic medical device (e.g., the heart for a prosthetic heart valve), retracting the capsule, in an axial direction, away from the prosthetic medical device and radially expanding the distal end portion so that the outer diameter of the distal end portion is equal to or larger than a diameter of the middle portion of the capsule.

In some embodiments, the distal end portion comprises an elastically deformable material and retracting the capsule away from the prosthetic heart valve and radially expanding the distal end portion so that the outer diameter of the distal end portion is equal to or larger than the diameter of the middle portion of the capsule at 306 includes elastically deforming the distal end portion radially outward, in response to a force of the prosthetic heart valve sliding axially outward from and through the distal end portion of the capsule.

In some embodiments, a proximal end portion of the capsule arranged on an opposite side of the middle portion from the distal end portion of the capsule is tapered radially inward from the middle portion. In these embodiments, during the retracting the capsule away from the prosthetic medical device at 306, the proximal end portion can remain tapered radially inward relative to a remainder of the capsule, including the middle portion and the distal end portion. Thus, in some embodiments, only the distal end portion of the capsule is configured to expand radially and the proximal end portion can remain tapered radially inward since the prosthetic medical device (e.g., valve) may exit the capsule from the distal end of the capsule (and does not need to pass through the proximal end of the capsule).

In this way, by configuring a capsule of delivery apparatus to have a narrowed (or tapered or smaller diameter) distal end portion, a narrowed proximal end portion, or both a narrowed distal end portion and proximal end portion, the amount of the outer surface of the capsule that can contact an inner surface of an introducer sheath and/or the patient's anatomy may be reduced. As explained herein, including these narrowed ends reduces the effective length of the capsule. As a result, potential degradation to the sheath or the patient's anatomy may be reduced and push forces experienced by a user of the delivery apparatus, during an implantation procedure, may be reduced. Thus, a user experience may be improved. Further, by forming the capsule of a flexible material and/or configuring the capsule to have elastically or plastically deformable wings (formed by spaced apart notches), the prosthetic medical device may be more easily loaded into the capsule and removed from the capsule, while still allowing the capsule to have a reduced effective length in the delivery configuration.

### Alternative Delivery Techniques

The preceding description describes a method for implanting a prosthetic valve using the disclosed delivery apparatuses to the native aortic valve via transfemoral delivery. However, it should be understood that the disclosed delivery apparatuses can be used to deliver a prosthetic valve (or another type of implantable medical device) to other native annulus of the heart (the pulmonary, mitral, and tricuspid valves), to vessels communicating with the heart (pulmonary artery, the inferior vena cava, or the superior vena cava), or to other locations within the body, using any of various delivery techniques.

For example, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) are introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature and/or an introducer sheath previously inserted into the patient's vasculature. Moreover, the disclosed delivery approaches are not intended to be limited. Any of the prosthetic valves disclosed herein can be implanted using any of various delivery procedures and delivery devices known in the art.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A capsule of a delivery apparatus configured to deliver a prosthetic medical device to a target implantation site, the capsule comprising: at least one end portion, arranged at a proximal end or distal end of the capsule, that narrows in diameter from a wider outer diameter of a middle portion of the capsule to a narrower outer diameter at an end of the at least one end portion, the end spaced away from the middle portion of the capsule, wherein the at least one end portion is configured to expand to the wider outer diameter in response to a radially outward pressure from the prosthetic medical device during removal of the prosthetic medical device from inside of the capsule.

Example 2. The capsule of any example herein, particularly example 1, wherein the at least one end portion is a distal end portion arranged at the distal end of the capsule.

Example 3. The capsule of any example herein, particularly example 1, wherein the at least one end portion is a proximal end portion arranged at the proximal end of the capsule.

Example 4. The capsule of any example herein, particularly example 1, wherein the at least one end portion is a distal end portion arranged at the distal end of the capsule and further comprising a proximal end portion arranged at the proximal end that narrows in diameter from the wider outer diameter to the narrower diameter at its proximal end.

Example 5. The capsule of any example herein, particularly example 4, wherein only the distal end portion is configured to expand to the wider outer diameter and the proximal end portion is configured to remain in its narrowed configuration.

Example 6. The capsule of any example herein, particularly any one of examples 1-5, wherein the at least one end portion comprises a plurality of elongate notches spaced apart from one another around a circumference of the at least one end portion and extending in an axial direction, from the end of the at least one end portion to a portion of the capsule that transitions from the at least one end portion to the middle portion of the capsule.

Example 7. The capsule of any example herein, particularly example 6, wherein the at least one end portion further comprises a plurality of wings formed by the plurality of notches, wherein each wing of the plurality of wings is formed between two adjacent notches of the plurality of notches.

Example 8. The capsule of any example herein, particularly example 7, wherein each wing is axially orientated, along a central longitudinal axis of the capsule, and angles radially inward from a wider, proximal end of the wing to a narrower, distal end of the wing, the distal ends of the plurality of wings forming the end of the at least one end portion of the capsule that is spaced away from the middle portion of the capsule.

Example 9. The capsule of any example herein, particularly any one of examples 6-8, wherein each notch includes an elongate end, extending from the end of the at least one end portion to the portion of the capsule that transitions from the at least one end portion to the middle portion of the capsule, and a bulbous end arranged at the portion of the capsule that transitions from the at least one end portion to the middle portion of the capsule.

Example 10. The capsule of any example herein, particularly any one of examples 1-9, wherein the capsule comprises an elastic, self-expanding metallic or polymeric material.

Example 11. The capsule of any example herein, particularly example 10, wherein the capsule comprises a shape memory material including a nickel titanium alloy.

Example 12. The capsule of any example herein, particularly any one of examples 1-11, wherein the at least one end portion of the capsule is configured to be plastically deformed or set into a delivery configuration where the at least one end portion is narrowed relative to the middle portion of the capsule such that the end of the at least one end portion has the narrower outer diameter.

Example 13. The capsule of any example herein, particularly any one of examples 1-9, wherein the at least one end portion is configured to elastically deform from a delivery configuration where the at least one end portion is narrowed relative to the middle portion of the capsule such that the end of the at least one end portion has the narrower outer diameter to an expanded configuration where the at least one end portion is expanded to the wider outer diameter.

Example 14. The capsule of any example herein, particularly any one of examples 1-9 and 13, wherein the at least one end portion of the capsule comprises an elastomeric material formed into a tapered shape that narrows in diameter from the wider outer diameter of the middle portion of the capsule to the narrower outer diameter at the end of the at least one end portion that is spaced away from the middle portion of the capsule.

Example 15. The capsule of any example herein, particularly any one of examples 1-14, wherein the capsule is configured to cover and enclose the prosthetic medical device therein in a radially compressed configuration and wherein the radially compressed prosthetic medical device comprises at least one tapered end that the at least one end portion is configured to surround.

Example 16. A delivery apparatus, comprising: an outer shaft; a nosecone coupled to a distal end of an inner shaft of the delivery apparatus, the inner shaft arranged within at least a portion of the outer shaft; and a capsule coupled to a distal end of the outer shaft, proximal to the nosecone, and configured to move axially relative to the nosecone, the capsule comprising: a cylindrical middle portion that has a first outer diameter along its length; and an end portion configured to move between a first configuration where the end portion tapers radially inward from the middle portion so that the end portion has a reduced diameter relative to the outer diameter of the middle portion and a second configuration where the end portion is expanded and has an expanded diameter that is larger than the reduced diameter.

Example 17. The delivery apparatus of any example herein, particularly example 16, wherein the expanded diameter is equal to or greater than the outer diameter of the middle portion.

Example 18. The delivery apparatus of any example herein, particularly any one of examples 16-17, wherein the end portion is a distal end portion of the capsule.

Example 19. The delivery apparatus of any example herein, particularly example 18, wherein the distal end portion includes a proximal end arranged adjacent to the middle portion and having an outer diameter equal to the outer diameter of the middle portion and wherein the distal end portion includes a distal end spaced away from the middle portion and having an outer diameter that is smaller than the outer diameter of the middle portion.

Example 20. The delivery apparatus of any example herein, particularly example 19, wherein the distal end of the distal end portion is configured to be arranged within an interior of a proximal end of the nosecone in a delivery configuration of the delivery apparatus, where the capsule covers a radially compressed prosthetic heart valve.

Example 21. The delivery apparatus of any example herein, particularly example 18, wherein the capsule further comprises a proximal end portion that has a same outer diameter as the outer diameter of the middle portion.

Example 22. The delivery apparatus of any example herein, particularly example 18, wherein the capsule further comprises a proximal end portion that tapers radially inward from the middle portion so that the proximal end portion has a reduced diameter relative to the outer diameter of the middle portion.

Example 23. The delivery apparatus of any example herein, particularly example 22, wherein when the distal end portion is in the second configuration, the proximal end portion remains tapered radially inward from the middle portion.

Example 24. The delivery apparatus of any example herein, particularly any one of examples 16-23, wherein the end portion comprises a plurality of elongate notches spaced apart from one another around a circumference of the end portion and extending in an axial direction, from an end of the end portion, the end spaced away from the middle portion, to a transition between the end portion and the middle portion of the capsule.

Example 25. The delivery apparatus of any example herein, particularly example 24, wherein the end portion further comprises a plurality of wings formed by the plurality of notches, wherein each wing of the plurality of wings is formed between two adjacent notches of the plurality of notches.

Example 26. The delivery apparatus of any example herein, particularly example 25, wherein each wing is axially orientated, relative to a central longitudinal axis of the capsule, and angles radially inward from a wider, proximal end of the wing to a narrower, distal end of the wing, the distal ends of the plurality of wings forming the end of the end portion of the capsule.

Example 27. The delivery apparatus of any example herein, particularly any one of examples 24-26, wherein each notch includes a wider end arranged at the transition between the end portion and the middle portion and an elongate end, extending between the wider end and the end of the end portion, wherein a width of the wider end is larger than a width of the elongate end.

Example 28. The delivery apparatus of any example herein, particularly any one of examples 16-27, wherein the capsule comprises an elastic, self-expanding metallic or polymeric material.

Example 29. The delivery apparatus of any example herein, particularly example 28, wherein the capsule comprises a nickel titanium alloy.

Example 30. The delivery apparatus of any example herein, particularly any one of examples 16-27, wherein the end portion of the capsule comprises an elastomeric material that is configured to elastically deform between the first configuration and the second configuration.

Example 31. The delivery apparatus of any example herein, particularly any one of examples 16-30, wherein a length of a portion of the capsule having a diameter that is equal to or larger than the outer diameter of the middle portion of the capsule is larger in the second configuration than in the first configuration.

Example 32. The delivery apparatus of any example herein, particularly any one of examples 16-31, wherein when the capsule is in the first configuration, the capsule is configured to cover and enclose a prosthetic heart valve therein, in a radially compressed configuration, and wherein the radially compressed prosthetic heart valve comprises at least one tapered end that the end portion is configured to surround.

Example 33. A method, comprising: in response to and during actuating a capsule of a delivery apparatus to move axially away from a radially compressed prosthetic medical device arranged on a distal end portion of the delivery apparatus, in order to uncover the radially compressed prosthetic medical device, radially expanding a distal end portion of the capsule from a narrowed configuration where an outer diameter of a distal end of the distal end portion is smaller than an outer diameter of a middle portion of the capsule to an expanded configuration where the outer diameter of the distal end is equal to or greater than the outer diameter of the middle portion.

Example 34. The method of any example herein, particularly example 33, wherein the radially expanding the distal end portion occurs while a proximal end portion of the capsule remains in a narrowed configuration where a proximal end of the proximal end portion has an outer diameter that is smaller than the outer diameter of the middle portion.

Example 35. The method of any example herein, particularly any one of examples 33-34, wherein the middle portion of the capsule is arranged between and adjacent to each of the distal end portion and a proximal end portion of the capsule.

Example 36. The method of any example herein, particularly example 35, wherein in the narrowed configuration, the distal end portion narrows in diameter, along its length, from a proximal end of the distal end portion that is arranged adjacent to the middle portion of the capsule to the distal end of the distal end portion.

Example 37. The method of any example herein, particularly any one of examples 35 and 36, wherein in the narrowed configuration, the proximal end portion narrows in diameter, along its length, from a distal end of the proximal end portion that is arranged adjacent to the middle portion of the capsule to a proximal end of the proximal end portion.

Example 38. The method of any example herein, particularly any one of examples 33-37, wherein the capsule is in the narrowed configuration during advancing at least the distal end portion of the delivery apparatus through an introducer sheath and an inner lumen of a patient, to a target implantation site for a prosthetic medical device arranged on the distal end portion of the delivery apparatus, within the capsule.

Example 39. The method of any example herein, particularly example 38, wherein the prosthetic medical device is a prosthetic heart valve.

Example 40. The method of any example herein, particularly any one of examples 33-39, wherein the radially expanding the distal end portion of the capsule includes separating distal ends of a plurality of wings spaced around a circumference of the distal end portion from one another and moving the distal ends of the plurality of wings radially outward.

Example 41. The method of any example herein, particularly example 40, wherein the separating and moving the distal ends of the plurality of wings radially outward includes widening a plurality of notches in the distal end portion, each notch of the plurality of notches arranged between two adjacent wings of the plurality of wings.

Example 42. The method of any example herein, particularly any one of examples 40 and 41, wherein the radially expanding the distal end portion of the capsule is responsive to a radially outward force applied to inner surfaces of the plurality of wings by an outer surface of the prosthetic medical device as the capsule is moved over a larger diameter portion of the outer surface of the prosthetic medical device and axially away from the prosthetic medical device.

Example 43. The method of any example herein, particularly any one of examples 40-42, wherein the radially expanding the distal end portion of the capsule includes elastically deforming the plurality of wings so that they move radially outward to the expanded configuration, in response to the radially outward force, and furthering comprising, returning the plurality of wings to the narrowed configuration after the radially outward force is removed, after moving the capsule away from the prosthetic medical device.

Example 44. A method, comprising: forming a tapered, narrower diameter portion of a capsule of a delivery apparatus at a distal end portion of the capsule, so that an outer diameter of the distal end portion narrows radially inward from a wider, middle portion of the capsule, the capsule arranged on a distal end portion of the delivery apparatus and configured to cover and retain a radially compressed prosthetic heart valve therein; and upon reaching a target implantation site for the prosthetic heart valve, retracting the capsule, in an axial direction, away from the prosthetic heart valve and radially expanding the distal end portion so that the outer diameter of the distal end portion is equal to or larger than a diameter of the middle portion of the capsule.

Example 45. The method of any example herein, particularly example 44, further comprising, after forming the tapered, narrower diameter portion of the capsule, advancing the distal end portion of the delivery apparatus to the target implantation site, through an introducer sheath and sliding an outer surface of the middle portion of the capsule along an inner surface of the introducer sheath while an outer surface of the distal end portion of the capsule remains spaced away from the inner surface of the introducer sheath.

Example 46. The method of any example herein, particularly any one of examples 44-45, wherein the forming the tapered, narrower diameter portion of the capsule includes moving distal ends of a plurality of wings forming the distal end portion of the capsule radially inward so that they are arranged proximate to one another and form the narrower diameter portion of the capsule, wherein each wing of the plurality of wings is separated from adjacent wings of the plurality of wings by an axially-extending notch in the distal end portion.

Example 47. The method of any example herein, particularly example 46, wherein forming the tapered, narrower diameter portion of the capsule further includes narrowing a plurality of notches arranged between and separating the plurality of wings, wherein each notch of the plurality of notches is arranged between two adjacent wings of the plurality of wings, and wherein radially expanding the distal end portion includes widening each notch of the plurality of notches.

Example 48. The method of any example herein, particularly any one of examples 46 and 47, wherein forming the tapered, narrower diameter portion of the capsule includes compressing the distal ends of the plurality of wings radially inward into a radially compressed configuration and setting the wings in the radially compressed configuration such that they are maintained in the radially compressed configuration until a radially outward force is applied to inner surfaces of the plurality of wings by the prosthetic heart valve, during the retracting the capsule.

Example 49. The method of any example herein, particularly any one of examples 46 and 47, wherein forming the tapered, narrower diameter portion of the capsule includes allowing the distal ends of the plurality of wings to retract radially inward into a non-deformed, resting state and, in response to retracting the capsule away from the prosthetic heart valve, elastically deforming the plurality of wings into an expanded state where distal ends of the plurality of wings are spaced further apart from one another around a circumference of the distal end portion.

Example 50. The method of any example herein, particularly any one of examples 44-45, wherein the distal end portion comprises an elastomeric material and wherein retracting the capsule away from the prosthetic heart valve and radially expanding the distal end portion so that the outer diameter of the distal end portion is equal to or larger than the diameter of the middle portion of the capsule includes elastically deforming the distal end portion radially outward, in response to a force of the prosthetic heart valve sliding axially outward from and through the distal end portion of the capsule.

Example 51. The method of any example herein, particularly any one of examples 44-50, wherein a proximal end portion of the capsule arranged on an opposite side of the middle portion from the distal end portion of the capsule is tapered radially inward from the middle portion and wherein during the retracting the capsule away from the prosthetic heart valve, the proximal end portion remains tapered radially inward relative to a remainder of the capsule, including the middle portion and the distal end portion.

Example 52. A method, comprising: forming a tapered, narrower diameter portion of a capsule of a delivery apparatus at a distal end portion of the capsule, so that an outer diameter of the distal end portion narrows radially inward from a wider, middle portion of the capsule, the capsule arranged on a distal end portion of the delivery apparatus and configured to cover and retain a radially compressed prosthetic heart valve therein; wherein the distal end portion of the capsule is configured to radially expand such that the outer diameter of the distal end portion, along a length of the distal end portion, is equal to or larger than a diameter of the middle portion of the capsule, in response to a radially outward pressure from the prosthetic heart valve during removal of the prosthetic heart valve from inside of the capsule.

In view of the many possible embodiments to which the principles of the disclosed technology may be applied, it should be recognized that the illustrated embodiments are only preferred examples of the disclosed technology and should not be taken as limiting the scope of the claimed subject matter. Rather, the scope of the claimed subject matter is defined by the following claims and their equivalents.

We claim:

1. A capsule of a delivery apparatus configured to deliver a prosthetic medical device to a target implantation site, the capsule comprising:
at least one end portion, arranged at a proximal end or distal end of the capsule, that narrows in diameter from a wider outer diameter of a middle portion of the capsule to a narrower outer diameter at an end of the at least one end portion, the end spaced away from the middle portion of the capsule, wherein the at least one end portion is configured to expand to the wider outer diameter in response to a radially outward pressure from the prosthetic medical device during removal of the prosthetic medical device from inside of the capsule.

2. The capsule of claim 1, wherein the at least one end portion is a distal end portion arranged at the distal end of the capsule.

3. The capsule of claim 1, wherein the at least one end portion is a proximal end portion arranged at the proximal end of the capsule.

4. The capsule of claim 1, wherein the at least one end portion is a distal end portion arranged at the distal end of the capsule and further comprising a proximal end portion arranged at the proximal end that narrows in diameter from the wider outer diameter to the narrower outer diameter at its proximal end.

5. The capsule of claim 4, wherein only the distal end portion is configured to expand to the wider outer diameter and the proximal end portion is configured to remain in its narrowed configuration.

6. The capsule of claim 1, wherein the at least one end portion comprises a plurality of elongate notches spaced apart from one another around a circumference of the at least one end portion and extending in an axial direction, from the end of the at least one end portion to a portion of the capsule that transitions from the at least one end portion to the middle portion of the capsule.

7. The capsule of claim 6, wherein the at least one end portion further comprises a plurality of wings formed by the plurality of notches, wherein each wing of the plurality of wings is formed between two adjacent notches of the plurality of notches, and wherein each wing is axially orientated, along a central longitudinal axis of the capsule, and angles radially inward from a wider, proximal end of the wing to a narrower, distal end of the wing, the distal ends of the plurality of wings forming the end of the at least one end portion of the capsule that is spaced away from the middle portion of the capsule.

8. The capsule of claim 6, wherein each notch includes an elongate end, extending from the end of the at least one end portion to the portion of the capsule that transitions from the at least one end portion to the middle portion of the capsule, and a bulbous end arranged at the portion of the capsule that transitions from the at least one end portion to the middle portion of the capsule.

9. The capsule of claim 1, wherein the capsule comprises an elastic, self-expanding metallic or polymeric material.

10. The capsule of claim 1, wherein the capsule is configured to cover and enclose the prosthetic medical device therein in a radially compressed configuration and wherein the prosthetic medical device in the radially compressed configuration comprises at least one tapered end that the at least one end portion is configured to surround.

11. A delivery apparatus, comprising:
an outer shaft;
a nosecone coupled to a distal end of an inner shaft of the delivery apparatus, the inner shaft arranged within at least a portion of the outer shaft; and
a capsule coupled to a distal end of the outer shaft, proximal to the nosecone, and configured to move axially relative to the nosecone, the capsule comprising:
a cylindrical middle portion that has a first outer diameter along its length; and
an end portion configured to move between a first configuration where the end portion tapers radially inward from the middle portion so that the end portion has a reduced diameter relative to the first outer diameter of the middle portion and a second configuration where the end portion is expanded and has an expanded diameter that is larger than the reduced diameter.

12. The delivery apparatus of claim 11, wherein the expanded diameter is equal to or greater than the first outer diameter of the middle portion.

13. The delivery apparatus of claim 11, wherein the end portion is a distal end portion of the capsule.

14. The delivery apparatus of claim 13, wherein the distal end portion includes a proximal end arranged adjacent to the middle portion and having an outer diameter equal to the outer diameter of the middle portion and wherein the distal end portion includes a distal end spaced away from the middle portion, the distal end having an outer diameter that is smaller than the outer diameter of the middle portion when in the first configuration.

15. The delivery apparatus of claim 14, wherein the distal end of the distal end portion, in the first configuration, is configured to be arranged within an interior of a proximal end of the nosecone in a delivery configuration of the delivery apparatus, where the capsule covers a radially compressed prosthetic heart valve.

16. The delivery apparatus of claim 13, wherein the capsule further comprises a proximal end portion that tapers radially inward from the middle portion so that the proximal end portion has a reduced diameter relative to the outer diameter of the middle portion and wherein when the distal end portion is in the second configuration, the proximal end portion remains tapered radially inward from the middle portion.

17. The delivery apparatus of claim 11, wherein the end portion comprises a plurality of elongate notches spaced apart from one another around a circumference of the end portion and extending in an axial direction, from an end of the end portion, the end spaced away from the middle portion, to a transition between the end portion and the middle portion of the capsule and wherein the end portion further comprises a plurality of wings formed by the plurality of notches, wherein each wing of the plurality of wings is formed between two adjacent notches of the plurality of notches and angles radially inward from a wider, proximal end of the wing to a narrower, distal end of the wing when the end portion is in the first configuration, distal ends of the plurality of wings forming the end of the end portion of the capsule.

18. The delivery apparatus of claim 11, wherein a length of a portion of the capsule having a diameter that is equal to or larger than the outer diameter of the middle portion of the capsule is larger in the second configuration than in the first configuration.

19. A method, comprising:
forming a tapered, narrower diameter portion of a capsule of a delivery apparatus at a distal end portion of the capsule, so that an outer diameter of the distal end portion narrows radially inward from a wider, middle portion of the capsule, the capsule arranged on a distal end portion of the delivery apparatus and configured to cover and retain a radially compressed prosthetic heart valve therein;
wherein the distal end portion of the capsule is configured to radially expand such that the outer diameter of the distal end portion, along a length of the distal end portion, is equal to or larger than a diameter of the middle portion of the capsule, in response to a radially outward pressure from the prosthetic heart valve during removal of the prosthetic heart valve from inside of the capsule.

20. The method of claim 19, wherein the forming the tapered, narrower diameter portion of the capsule includes moving distal ends of a plurality of wings forming the distal end portion of the capsule radially inward so that they are arranged proximate to one another and form the narrower diameter portion of the capsule, wherein each wing of the plurality of wings is separated from adjacent wings of the plurality of wings by an axially-extending notch in the distal end portion.
